(19) United States
(12) Patent Application Publication
(10) Pub. No.: US 2008/0268011 A1
(43) Pub. Date: Oct. 30, 2008

(54) Antimicrobial Implant with a Flexible Porous Structure

(76) Inventors: Helmut Goldmann, Tuttlingen/Donau (DE); Dennis Langanke, Tuttlingen/Donau (DE); Volker Friedrich, Neuhausen (DE)

Correspondence Address: NATH & ASSOCIATES, 112 South West Street, Alexandria, VA, 22314, US

(21) Appl. No.: 11/663,595

(22) PCT Filed: Sep. 22, 2005
(86) PCT No.: PCT/EP2005/010256

§ 371 Date: Mar. 21, 2008

(30) Foreign Application Priority Data
Sep. 24, 2004 (DE) 10 2004 047 568.7

(51) Int. Cl. A61F 2/02

(52) U.S. Cl. 424/423

(57) ABSTRACT

The invention relates to an antimicrobial implant with a flexible porous structure composed of a biocompatible synthetic material which comprises particles of at least one antimicrobial agent.

The invention relates to an antimicrobial implant with a flexible porous structure composed of a biocompatible synthetic material.

There has recently been an increasing trend to provide implants, such as vascular prostheses, with an anti-microbial finish in order to suppress primary infections or else secondary infections. Thus, U.S. Pat. No. 5,019,096 describes a method for making a wide variety of types of implants resistant to infection. To this end, the implants or other medical articles such as catheters and rubber gloves are provided on their surface with a coating material which comprises synergistic amounts of a silver salt and of a biguanide.

EP 0 184 465 discloses a thermoplastic polyurethane product in which at least one layer of a thermoplastic polyurethane is provided on a substrate and which may comprise antithrombogenic materials and antibiotic materials in order to avoid thromboses and infections where appropriate after implantation.

DE 100 43 151 A1 describes a bone cement with antimicrobial activity. This activity is generated by mixing nano- to microdisperse silver in amounts of up to 2% by weight in the cement material. DE 43 44 306 A1 describes an article of synthetic material which has metallic silver on and/or underneath the surface. The article, e.g. a tubing, may have a coating of metallic silver. It is also possible to incorporate silver powder into compositions for extrusion. WO 2004/060210 A1 discloses the production of radiopaque vascular prostheses by mixing PTFE particles before they are processed to prostheses of expanded PTFE with particles of radiopaque material, e.g. silver. Apart from the fact that there is a risk here of release of silver particles into the bloodstream, because the silver particles are not embedded in the PTFE particles themselves, the possibilities of variation are limited.

WO 96/03165 A1 describes hernia implants with a porous structure in the form of a microporous membrane or of a mesh fabric. The porous structure may be impregnated with a resorbable material which comprises an antimicrobial agent.

In EP 0 633 032 B1, a vascular prosthesis composed of a tubular porous article is provided with an antibacterial finish by winding a tube, fiber or sheet composed of a polymeric material which is provided with an antibacterial substance around the vascular prosthesis in such a way that part of the surface of the vascular prosthesis remains uncovered.

In WO 98/31404, prostheses composed of biological tissue or biological polymer are provided with an antimicrobial finish by providing the prosthesis with exogenous material which comprises silver ions.

The invention is based on the object of producing an antimicrobial implant with a flexible porous structure composed of a biocompatible synthetic material in a simple manner in such a way that a long-lasting antimicrobial effect is attained. This is achieved by the antimicrobial agent being present in the synthetic material of the implant.

The invention thus relates to an antimicrobial implant with a flexible porous web structure composed of a biocompatible synthetic material which comprises particles of at least one antimicrobial agent. Due to the fact that the synthetic material comprises the antimicrobial agent, i.e. inside the fibers of the fiber web, production of the porous implant is possible by known techniques without the need subsequently to apply special coatings. A substantial advantage of the invention is also that the pores of the implant of the invention are unoccupied, i.e. not blocked by any coating materials which comprise the antimicrobial agent or by the agent particles themselves. The implant of the invention can also be handled and processed further in a conventional way. Thus, it can be sterilized by conventional sterilization methods, in particular with ethylene oxide.

The implant of the invention may be designed as a planar implant, for example as patch, in particular for covering or closing wounds. It may thus be designed as hernia implant. The implant is also advantageously designed as hollow organ to replace hollow organs of the human or animal body. Thus, the implant of the invention is designed in a preferred embodiment of the invention as vascular prosthesis. Hollow organs suitable according to the invention are defined as flexible porous tubes, in particular with an internal diameter of from 2 to 38 mm. Small-lumen tubes with an internal diameter of from 2 to 24 mm, in particular from 2 to less than 14 mm, prove to be particularly suitable. The porous structure is preferably a microporous structure, in particular one with an air permeability of from 5 to 500 ml/cm²/minute with a pressure difference of 1.2 kPa. The porosity of the, in particular three-dimensional, structure can be adjusted by suitable production processes. Thus, preferred ranges of porosity in the range from 10 to 200 ml, in particular 10 to 100 ml or 100 to 200 ml/cm²/minute can be adjusted.

The biocompatible synthetic material is advantageously a non-absorbable synthetic material. The implant of the invention is thus long-lasting. It has emerged that the synthetic material can release sufficient amounts of antimicrobial agent to display the antimicrobial efficacy even if it is not degradable. Advantageously suitable as biocompatible synthetic material are thermally plasticizable synthetic materials, especially thermoplastics. Synthetic materials which are soluble in organic solvents are also suitable. Thus, synthetic materials which are advantageously suitable are polyesters, e.g. Dacron®, polypropylene, polyvinylidene fluoride, polyamide, polyetherketones, polytetrafluoroethylene and polyurethane. Synthetic materials with hydrophobic properties are preferred for certain applications, such as polyurethane and polypropylene.

The implant of the invention is preferably designed with thin walls and has in particular a wall thickness of from 0.1 to 2 mm, preferably 0.3 to 0.6 mm.

According to the invention, the flexible porous web structure is formed by a wall material which is a nonwoven, i.e. neither a knitted material nor a woven material. A fiber web produced by material application, in particular a sprayed web or a meltblown web, is as particularly advantageous. The processes for producing such webs are known.

The particles of the at least one antimicrobial agent may advantageously be dispersed substantially homogeneously in the synthetic material. The homogeneity preferably relates to the synthetic material of each individual fiber of the web structure. Depending on the location of the fibers, they may have a different agent concentration than fibers at another location in the web structure. It may desired in particular embodiments for the particles of the at least one antimicrobial agent to be enriched on at least one surface of the implant. Configurations of this type can easily be generated with the production techniques available here, for example the spraying technique. It is possible in the production of the web structure to operate with different solutions or melts of synthetic material which differ in particular by the concentration of the particles of antimicrobial agent. The solutions or melts can be discharged from different spray guns. A layered structure of the implants is possible thereby. The different concentrations may be brought about by different amounts of particles and/or by different sizes of the particles in the synthetic material.

The size of the particles with an antimicrobial effect is preferably in the nanometer range. Particles sizes of from 50 to 100 nm are particularly suitable. The particles may be linked to form agglomerates which may have in particular a size of from 5 to 10 μm. Particularly suitable as material for the particles with an antimicrobial effect are metals or metal alloys with an antimicrobial effect, with particular preference for metallic silver. The outstanding antimicrobial effect of silver is well known. Suitable silver particles are described for example in WO 02/17984 A1. The content of particles of the at least one antimicrobial agent in the synthetic material of the implant of the invention may vary within wide limits. The content is preferably from 0.1 to 10% by weight, in particular 0.2 to 5% by weight. Amounts of from 0.5 to 2.5% by weight are usually particularly suitable.

The invention also relates to a process for producing the implant of the invention. In the process, the synthetic material is treated with the particles of the antimicrobial agent at the latest during, and preferably before, the shaping of the porous web structure of the implant. The implant is preferably formed from the synthetic material which is provided with the particles of the at least one antimicrobial agent in one operation by material application to a substrate, in particular by a spraying technique. As already mentioned, the sprayed web technique is particularly suitable as spraying technique. Solutions of the biocompatible synthetic material which already comprise the particles of the at least one antimicrobial agent can be processed without modifying the spraying technique. The agent particles, especially in the form of nanoparticles, can be dispersed homogeneously in the solution. Especially for the production of the flexible porous web structure on application of the meltblown web technique it is possible to incorporate the at least one agent also in the melt of the synthetic material.

Further features and advantages of the invention are evident from the following description of preferred examples in conjunction with the dependent claims. It is possible in this connection for the individual features each to be implemented on its own or together with one another in combination.

Production of a Polyurethane Vascular Prosthesis

1200 g of polyurethane granules and 13 500 g (9 liters) of chloroform are put into a stirred vessel and stirred at about 260 rpm for 72 hours until dissolution is complete.

Then 73.5 g of nanosilver with a particle size of about 50 to 100 nm (agglomerated 5 to 10 μm) are added to the solution and homogenized by stirring (3 h). The resulting suspension is sprayed through two spray guns (e.g. Krautzberger type A-7) with compressed air at 3.5 bar. The guns are disposed a distance of 350 mm apart and at an angle of 60° to a roll rotating at 300 rpm. Spraying is carried out firstly for 150 cycles with the guns at a distance of 23.5 cm from the roll and then for 280 cycles at a distance of 11 cm from the roll and finally once again for 100 cycles at a distance of 23.5 cm. It is also possible to operate with suspensions differing in silver content, especially in the different cycles. The silver content may in particular be higher on at least one surface of the prosthesis than inside the prosthesis wall. After completion of the spraying process, roll and web are immersed in an organic solvent for 10 seconds so that the web is completely wetted. After a dripping time of 5 min, the web is rotated while drying. The resulting vascular prosthesis has an internal diameter of 6 mm and an external diameter of 7 mm. It has a slight gray coloration.

The silver content of a vascular prosthesis produced in this way is 6.1±0.4% by weight. In just the same way a preset silver content of the spraying solution results in the following silver contents of the prosthesis:

Silver content of the spraying solution 0.1%→1.2% by weight silver content of the prosthesis

Silver content of the spraying solution 0.5%→6.1% by weight silver content of the prosthesis

Silver content of the spraying solution 1.0%→12.3% by weight silver content of the prosthesis

Porosity and Pore Size of PU Prosthesis
Porosity 50% (free volume)
Pore size 95% of the pores have a pore size between 0.1 and 100 μm. The majority (more than 50% of the pores) has a size between 1 and 10 μm. The commonest pore size is 1 μm.

Water Penetration Pressure
Method: The test prostheses are filled with water and exposed to increasing pressure. As soon as water is observed on the external surface, the pressure is recorded and the test is stopped. This pressure is the water penetration pressure. The water penetration pressure of the PU vascular prostheses is 250 to 320 mmHg.

2. Test of Antimicrobial Activity

1) Test Method

The test specimens are incubated with cells of the test strain, and the proliferation of daughter cells to the surroundings is ascertained via the optical density.

Test Strain

Staphylococcus Epideridis 9142 (˜10⁶ CFU/ml)

2) Procedure

The vascular prostheses were cut into test specimens 7×4 mm in size and investigations were carried out on a number of n=8 per test specimen.

The test was moreover carried out once with and once without preincubation in human plasma. On preincubation in human plasma, the samples were incubated therein at 37° C. for 2 h and then washed with phosphate buffer solution.

All the test specimens were incubated with the cells of the test strain initially at 37° C. for 1 hour in order to achieve complete adhesion of the cells to the surface of the test specimen. The organisms not adhering to the surface were rinsed off by a single washing in phosphate buffer solution. The test specimens were then incubated with the cells of the test strain at 37° C. for a further 24 hours for the antimicrobial properties to be displayed, and were then removed from the incubation medium.

The proliferated daughter cells in the remaining bacterial suspension were ascertained optically over a period of 48 hours, with the time necessary to exceed a particular threshold value being indicated as growth delay in hours.

3) Material

Polyurethane prostheses with silver incorporated.

The contents of added nanosilver based on the initial PU/chloroform solution were 0.1%, 0.5% and 1.0% (percent by weight based on PU 1.2%, 6.1%, 12.3%).

4) Results

The growth delay of the silver-containing test samples emerges from the measured time difference of the silver-containing test specimens from the reference value of the non-silver-containing test specimen.

Without Preincubation

| Sample | | |
|---|---|---|
| designation | Growth delay | |
| (Ag % by weight) | (h) | Result |
| PU control | 3.9 | not antibacterial |
| PU 0.1% | 14.7 | antimicrobial |
| PU 0.5% | 20.5 | antimicrobial |
| PU 1.0% | 33.2 | antimicrobial |

With Preincubation in Human Plasma

| Sample | | |
|---|---|---|
| designation | Growth delay | |
| (Ag % by weight) | (h) | Result |
| PU control | 3.9 | not antimicrobial |
| PU 0.1% | 16.6 | antimicrobial |
| PU 0.5% | 27.0 | antimicrobial |
| PU 1.0% | 32.1 | antimicrobial |

Both without and with additional preincubation in human plasma, proliferated daughter cells of the test strain were detected after only a short time with the non-silver-containing polyurethane prostheses, whereas marked time delays of growth occur with the prostheses with additional silver incorporation.

The antimicrobial activity in these cases depends on the silver concentration and thus increases in the sequence 0.1%<0.5%<1.0%.

1-19. (canceled)

20. An antimicrobial implant with a flexible porous web structure composed of a biocompatible synthetic material which comprises particles of at least one antimicrobial agent.

21. The implant as claimed in claim 20 wherein the implant is designed as hollow organ.

22. The implant as claimed in claim 20, wherein the implant is designed as planar implant.

23. The implant as claimed in claim 20, wherein the porous structure is a microporous structure.

24. The implant as claimed in claim 20, wherein the synthetic material is a non-absorbable synthetic material.

25. The implant as claimed in claim 20, wherein the synthetic material is a thermally plasticizable synthetic material.

26. The implant as claimed in claim 20, wherein the synthetic material is a synthetic material which can dissolve in solvents.

27. The implant as claimed in claim 20, wherein the synthetic material is a hydrophobic synthetic material.

28. The implant as claimed in claim 20, wherein the implant is designed with thin walls.

29. The implant as claimed in claim 20, wherein the implant is designed as a sprayed web.

30. The implant as claimed in claim 20, wherein the implant is designed as melt-blown web.

31. The implant as claimed in claim 20, wherein the particles of the antimicrobial agent are homogeneously dispersed in the synthetic material of single fibers of the web structures.

32. The implant as claimed in claim 20, wherein the particles of the antimicrobial agent are enriched on at least one surface of the implant.

33. The implant as claimed in claim 32, wherein fibers of the web structure located on the surface have a higher concentration of particles of the antimicrobial agent than those fibers located underneath the surface.

34. The implant as claimed in claim 20, wherein the particles of the at least one antimicrobial agent have a size of from 50 to 100 ran.

35. The implant as claimed in claim 20, wherein the particles of the at least one antimicrobial agent consist of at least one metal with antimicrobial activity.

36. The implant as claimed in claim 20, wherein the particles of the at least one antimicrobial agent are present with a content from 0.1 to 10% by weight.

37. A process for producing the implant as claimed in claim 20 wherein biocompatible synthetic material is treated with particles of at least one antimicrobial agent at the latest during the shaping of the porous structure of the implant.

38. The process as claimed in claim 37, wherein the implant composed of the synthetic material which is provided with the particles of the at least one antimicrobial agent is formed in one operation.

39. An antimicrobial implant with a flexible porous web structure composed of a biocompatible synthetic material which comprises particles of at least one antimicrobial agent wherein the implant is designed as vascular prosthesis.

40. The implant as claimed in claim 22, wherein the implant is designed as patch.

41. The implant as claimed in claim 23, wherein the microporous structure has an air permeability of from 5 to 500 mUcm²/minute with a pressure difference of 1.2 kPa.

42. The implant as claimed in claim 28, wherein the walls have a wall thickness of from 0.1 to 2 mm.

43. A process for producing the implant as claimed in claim 37, wherein the biocompatible synthetic material is treated with the particles of the at least one antimicrobial agent before the shaping of the porous structure of the implant.

44. The process as claimed in claim 38, wherein the implant is formed by material application to a substrate.
